(19) United States
(12) Patent Application Publication
(10) Pub. No.: US 2005/0002900 A1
(43) Pub. Date: Jan. 6, 2005

(54) Method of treating estrogen responsive breast cancer

(76) Inventors: Grace Wong, Brookline, MA (US); Aliza Eshkol, Rippe (CH); Giampiero DeLuca, Chemin de la Florence (CH)

Correspondence Address: David S Resnick;Nixon Peabody, 100 Summer Street, Boston, MA, 02110-2131, US

(21) Appl. No.: 10/494,644

(22) PCT Filed: Nov. 5, 2002
(86) PCT No.: PCT/US02/35438

Related U.S. Application Data
(60) Provisional application No. 60/332,939, filed on Nov. 6, 2001.

(51) Int. Cl. A61K 38/21; A61K 39/395

(52) U.S. Cl. 424/85.5; 424/145.1

(57) ABSTRACT

The present invention is directed to a method of treating estrogen responsive breast cancer in an individual comprising administering to an individual a therapeutically effective estradiol inhibiting amount of interferon gamma (IFN-γ) and/for a tumor necrosis factor (TNF) antagonist and/or an interleukin-1 (IL-1) antagonists. The invention is based upon the surprising discovery that IFN-γ and/or a tumor necrosis factor (TNF) antagonist and/or an interleukin-1 (IL-1) antagonists inhibit estradiol production in human adipocytes. This discovery is not only important because it allows for the treatment and/or prevention of estrogen dependent breast cancer using IFN-γ, TNF antagonists or IL-1 antagonist each alone or in combination, but also because IFN-γ and/or TNF antagonists and/or IL-1 antagonists can be used in conjunction with standard anti-estrogen therapy, e.g., tamoxifen and/or aromatase inhibitor, to result in lower estrogen levels than seen with standard anti-estrogen therapy alone. Moreover, the ability to lower estrogen levels by means of the present invention, when combined with standard anti-estrogen therapy, provides an important therapeutic option in that it allows the dose of the anti-estrogen to be reduced, reducing the likelihood of side effects and complications commonly seen with anti-estrogen therapy.

### BACKGROUND OF THE INVENTION

Breast cancer is a significant health problem for women in the United States and throughout the world. Although advances have been made in detection and treatment of the disease, breast cancer remains the second leading cause of cancer-related deaths in women, affecting more than 180,000 women in the United States each year (Forbes, Seminars in Oncology, vol.24(1), Suppl 1, 1997: pp.S1-20-S1-35). It is currently estimated that in the United States women have a one in eight chance of developing the disease in their lifetime (by the age of eighty), whereas one in twenty-eight women have a lifetime risk of dying from breast cancer (Harris et.al., Ed. Diseases of the Breast, 1996: pp. 159-168).

Although male breast cancer is rare, it still accounts for about 1% of all breast carcinomas (Borgen P I, Wong G Y, Vlamis V, et al.: Current management of male breast cancer: a review of 104 cases. Annals of Surgery 215(5): 451-459, 1992). The mean age at diagnosis is between 60 and 70, although men of all ages can be affected with the disease (Jaiyesimi I A, Buzdar A U, Sahin A A, et al.: Carcinoma of the male breast. Annals of Internal Medicine 117(9): 771-777, 1992). Predisposing risk factors appear to include radiation exposure, estrogen administration, and diseases associated with hyperestrogenism, such as cirrhosis or Klinefelter's syndrome (Hultborn R, Hanson C, Kopf I, et al.: Prevalence of Klinefelter's syndrome in male breast cancer patients. Anticancer Research 17(6D): 4293-4298, 1997). There are also definite familial tendencies, with an increased incidence seen in men who have a number of female relatives with breast cancer.

Historically, the first to discover the role played by endocrine treatment in breast cancer was Beatson, in 1896, who observed that breast cancer in pre-menopausal women undergoes remission after oophorectomy. This finding, subsequently confirmed by other scientists, supported the evidence that at least some breast tumors are directly dependent on hormones for their growth and created interest in the therapeutic approach of endocrine organ ablation for the purpose of removing the endogenous source of hormones. In the surgical endocrinotherapy for breast cancer, an organ involved in the secretion of estrogen is removed to regress estrogen dependent breast cancer. This, however, results in loss of not only estrogen but also life-sustaining hormones, including steroid hormones, posing many problems associated with the quality of life.

It is now known that estrogen plays a major role in the development of breast cancer. Approximately 60% of premenopausal and 75% of postmenopausal patients have estrogen-dependent tumors. In men, over 80% of breast tumor tissues contain hormone receptors and about two-thirds of men respond to hormonal therapy. (Richard B. Everson and Marc E. Lippman. “Male Breast Cancer,” in Breast Cancer, Volume 3. William L. McGuire, ed. New York: Plenum Plublishing Corporation, 1979).

During the treatment of estrogen-dependent breast cancer, it would be important to greatly reduce or, if possible, eliminate estrogen-induced effects. For this purpose, it is desirable both to block receptor sites stimulated by estrogen and also to reduce the amount of estrogen to act at these sites.

In addition to endocrine ablation in the form of ovarectomy, current therapy of estrogen dependent breast cancer relies on the use of anti-estrogen compounds such as, for example, tamoxifen. However, these anti-estrogenic agents may have stimulatory effects on certain cancer cell populations in the uterus due to their estrogenic (agonist) properties and they may, therefore, be counterproductive in some cases. One reason is that endogenous hormone production implicates a hyper-activity of the hypothalamic-pituitary-gonadal axis.

For example, the most serious side effect is tamoxifen's estrogenic effect in the uterus which causes endometrial hyperplasia and a substantial increase in the incidence of endometrial carcinomas (a three to four-fold increase in risk after five years tamoxifen administration) (Goldhirsch etal., Endocrine Therapies of Breast Cancer, Sem in Onc, vol.23(4), pp.494-505, 1996). For this reason and the lack of improvement in survival advantage with long-term tamoxifen use, tamoxifen therapy of longer than five years is now contraindicated. In addition, tamoxifen is also associated with a significantly increased incidence of venous thromboembolism, substantially increased incidence of vasomotor symptoms or hot flashes (in the range of 16-67%), cataract formation, and DNA-adduct formation which, although not clinically confirmed, still raises concerns about the potential for hepatocellular carcinoma (observed experimentally in animal models). Moreover, data suggest that with long-term tamoxifen exposure, breast tumor cells undergo alterations that cause them to develop resistance to its antiestrogenic effects, and alternatively respond to its estrogenic properties (Santen, Editorial: Long Term Tamoxifen Therapy: Can an Antagonist become an Agonist?, J Clin Endo & Metab, vol. 81(6), pp.2027-2029, 1996).

Thus, although such mixed estrogen agonist-antagonists, like tamoxifen, have beneficial effects in the treatment of estrogen dependent breast cancer, and the estrogenic side-effects are tolerable in acute life-threatening situations, they are not ideal.

Therefore, while surgical intervention and anti-estrogen therapies are available for estrogen dependent breast cancer, it would be useful to have an agent which alone or in combination with the other anti-estrogenic agents would eliminate or reduce the side effects of currently used anti-estrogen therapies.

### SUMMARY OF THE INVENTION

The present invention is based on the surprising discovery that interferon gamma (IFN-γ) and/or a tumor necrosis factor (TNF) antagonist and/or an interleukin-1 (IL-1) antagonists inhibit estradiol production in human adipocytes. This discovery is not only important because it allows for the treatment and/or prevention of estrogen dependent breast cancer using IFN-γ, TNF antagonists or IL-1 antagonist each alone or in combination, but also because IFN-γ and/or TNF antagonists and/or IL-1 antagonists can be used in conjunction with standard anti-estrogen therapy, e.g., tamoxifen and/or aromatase inhibitor, to result in lower estrogen levels than seen with standard anti-estrogen therapy alone. Moreover, the ability to lower estrogen levels by means of the present invention, when combined with standard anti-estrogen therapy, provides an important therapeutic option in that it allows the dose of the anti-estrogen to be reduced, reducing the likelihood of side effects and complications commonly seen with anti-estrogen therapy.

In one embodiment, a method is provided herein to treat and/or prevent estrogen responsive breast cancer in an individual an comprising of administration of a therapeutically effective estradiol inhibiting amount of IFN-γ and/or one or more TNF antagonists and/or one or more IL-1 antagonists.

In a second embodiment, the invention provides a method for treating and/or preventing estrogen responsive breast cancer in an individual comprising administering a therapeutically effective estradiol inhibiting amount of IFN-γ and/or one or more TNF antagonists and/or one or more IL-1 antagonists in combination with an anti-estrogenic agent Tamoxifen and/or an aromatase inhibitor is a preferred anti-estrogenic agent.

The present invention further provides the use of IFN-γ and/or one or more TNF antagonists and/or one or more IL-1 antagonists alone or in combination together with a pharmaceutical acceptable carrier in the preparation of pharmaceutical compositions for treatment and/or prevention of estrogen responsive breast cancer. In an alternative embodiment, the composition includes a standard anti-estrogenic agent. Tamoxifen and/or an aromatase inhibitor is a preferred anti-estrogenic agent.

In the present invention, the routes of administration of IFN-γ and/or TNF antagonists and/or IL-1 antagonists include intradermal, transdermal (e.g. in slow release formulations), intramuscular, intraperitoneal, intravenous, subcutaneous, oral, epidural, topical, and intranasal routes. The preferred route of administration is parenteral. Any mode of parenteral administration may be suitable including intravenous, intramuscular and subcutaneous. Besides the pharmaceutically acceptable carrier, the composition of the invention can also comprise minor amounts of additives, such as stabilizers, excipients, buffers and preservatives.

IFN-γ useful in the method of the present invention includes native IFN-γ, recombinant IFN-γ and IFN-γ that has been modified to, for example, increase its stability.

TNF antagonists useful in the method of present invention include soluble TNF receptor molecules, anti-TNF antibodies and compounds which prevent and/or inhibit INF receptor signaling. Proteins, muteins, protein-derived peptides, mimetics and small molecule drugs that inhibit estradiol induction by TNF in adipocytes can also be used in the present invention. It is possible to use the TNF antagonists alone or a combination with other TNF antagonists.

Specific TNF antagonists useful in the method of the present invention include recombinant human TBP (r-h TBP, tumor necrosis factor binding protein described e.g. in U.S. Pat. No. 6,225,300) from Serono S A; Etanercept (ENBREL™) from Immunex Corporation; Infliximab (REMICADE™) from Centocor, Inc.; Ienercept (RO-45-2081, Tenefuse) from Hoffman-La Roche; soluble TNF Receptor Type I (sTNF-R1 from Amgen Inc.); other agents containing soluble TNF receptors; CDP571 (a humanized monoclonal anti-TNF alpha antibodies) from Celltech Chiroscience; other monoclonal anti-TNF alpha antibodies; D2E7/adalimumab (a human anti-TNF mAb) from Abbott; thalidomide; phosphodiesterase 4 (IV) inhibitor thalidomide analogues; other phosphodiesterase IV inhibitors; and TNF alpha converting enzyme.

The IL-1 antagonists include IL-1 receptor antagonists, IL-1 binding proteins, anti-IL-1 monoclonal antibodies, IL-1 receptor accessory proteins and other compounds and proteins which block in vivo synthesis or extracellular release of IL-1. In the preferred embodiment, the IL-1 antagonist is an IL-1β antagonist.

The invention also features an article of manufacture including packaging material and a pharmaceutical agent contained therein that is therapeutically effective for treating or preventing estrogen-dependent breast cancer in an individual. The packaging material may include a label that indicates that the pharmaceutical agent can be used for treating and/or preventing estrogen-dependent breast cancer. The pharmaceutical agent includes an effective amount of IFN-γ or an effective amount of a TNF antagonist or an effective amount of IL-1 antagonist. In one embodiment, the pharmaceutical agent includes a combination of an effective amount of IFN-γ and/or a TNF antagonist and/or an IL-1 antagonist. In another embodiment, the invention includes a combination of IFN-γ, a TNF antagonist and an IL-1 antagonist.

In an alternate embodiment, the invention relates to compositions and kits comprising a first estrogen-dependent cancer treating or preventing agent including IFN-γ, a TNF antagonist and/or an IL-1 antagonist, alone or in combination, and a second therapeutic agent. The second therapeutic agent is not IFN-γ or a TNF antagonist or an IL-1 antagonist. These compositions are effective to treat or prevent estrogen-dependent breast cancer in an individual. Various classes of therapeutic agents, including anti-estrogenic agents such as tamoxifen, may be used in the composition. In one embodiment, the first estrogen-dependent cancer treating or preventing pharmaceutical agent includes a combination of an effective amount of IFN-γ and a TNF antagonist and/or IL-1.

### BRIEF DESCRIPTION OF THE FIGURES

FIG. 1 shows athat IFN-γ inhibits tumor necrosis factor alfa (TNF-α) and tumor necrosis factor beta (TNF-β) induced estradiol production in human adipocytes.

FIG. 2 shows that interferon gamma (IFN-γ) inhibits the transcription of aromatase mRNA when transcription is induced by interleukin-1β (IL-1β) or TNF. The lower panel shows transcription of a household gene, glyceraldehyde-phosphate dehydrogenase (GAPDH). The level of estradiol regulated by IL-1β or TNF±IFN-γ are also shown in pg/ml. Control lane shows the base level of aromatase mRNA expression in adipocytes.

FIG. 3 shows differential effects of interferon (IFN) α, β, and γ on the expression of aromatase MnRNA in human adipocytes. The estradiol production is induced by about 6 fold upon IFN-α induction and by about 16 fold by IFN-β induction and blocked completely by IFN-γ.

FIG. 4 shows that activation of TNF-receptor in human fat cells is responsible for estradiol production and that a soluble TNF binding protein, TNF-R1 (TBP), completely blocks TNF-α and TNF-β induced estradiol production.

It is to be understood that both the foregoing general description and the following detailed description are merely exemplary of the invention, and are intended to provide an overview or framework for understanding the nature and character of the invention as it is claimed.

### DETAILED DESCRIPTION OF THE INVENTION

The invention described herein clearly shows the unexpected result that estradiol production from human adipocytes is reduced by blocking TNF by a TNF antagonist, an IL-1 antagonist or by addition of IFN-γ. While not wishing to be bound by theory, it is believed that the reduction in estradiol production is the result of inhibition of induction of aromatase mRNA. As a result, one embodiment of the present invention provides a method to treat and/or prevent estrogen responsive breast cancer in an individual comprising administering a therapeutically effective estradiol production inhibiting amount of IFN-γ and/or a TNF antagonist and/or an IL-1 antagonist alone or in combination.

In another embodiment, the invention relates to a method for treating or preventing estrogen responsive breast cancer in an individual comprising administration of a therapeutically effective estradiol production inhibiting amount of IFN-γ and/or a TNF antagonist and/or an IL-1 antagonist, alone or in combination, and in combination with an anti-estrogenic agent. Preferably, the anti-estrogenic agent is non-steroidal. Tamoxifen is a preferred anti-estrogen agent

A further embodiment of the present invention is the use of IFN-γ and/or a TNF antagonist and/or IL-1 antagonist alone or in combination together with a pharmaceutically acceptable carrier in the preparation of pharmaceutical compositions for the treatment and/or prevention of estrogen responsive breast cancer in an individual. The pharmaceutical composition may also include an anti-estrogenic agent.

The active ingredients used in the present invention are TNF antagonists, IL-1 antagonists, IFN-γ and anti-estrogenic agents. TNF antagonists exert their activity in one of two ways. First, antagonists can bind to or sequester the TNF molecule itself with sufficient affinity and specificity to substantially neutralize the TNF epitope responsible for TNF receptor binding (hereinafter termed sequestering antagonists). Alternatively, TNF antagonists can inhibit TNF signaling pathway activated by the cell surface receptor after TNF binding (hereinafter termed “signaling antagonists”). Both groups of antagonists are useful, either alone or together, in the therapy of estrogen response breast cancer, according to the present invention.

TNF antagonists are easily identified and rated by routine screening of candidates for their effect on the activity of native TNF on susceptible cell lines in vitro, for example human B cells, in which TNF causes proliferation and Ig secretion. The assay contains TNF formulation at varying dilutions of candidate antagonist, e.g. from 0.1 to 100 times the molar amount of TNF used in the assay, and controls with no TNF or only antagonist (Tucci et al., Effects of eleven cytokines and of IL-1 and tumor necrosis factor inhibitors in a human B cell assay. J Immunol. 1992 May 1;148(9):2778-84).

Sequestering antagonists are the preferred TNF antagonists according to the present invention. Among sequestering antagonists, those polypeptides that bind TNF with high affinity and possess low imnmunogenicity are preferred. Soluble TNF receptor molecules and neutralizing antibodies to TNF are particularly preferred. For example, TNF-RI and TNF-RH are useful in the present invention. Truncated forms of these receptors, comprising the extracellular domains of the receptors or functional portions thereof, are more particularly preferred antagonists according to the present invention. Truncated forms of the TNF receptors are soluble and have been detected in urine and serum as 30 kDa and 40 kDa TNF inhibitory binding proteins, which were originally called respectively TBPI and TBPII (Engelmann et al., Two tumor necrosis factor-binding proteins purified from human urine. Evidence for immunological cross-reactivity with cell surface tumor necrosis factor receptors. J Biol Chem. 1990 Jan 25;265(3):1531-6.). Derivatives, fragments, regions and biologically active portions of the receptor molecules functionally resemble the receptor molecules that can be used in the present invention. Such biologically active equivalent or derivative of the receptor molecule refers to the portion of the said polypeptide, or of the sequence encoding the receptor molecule, that is of sufficient size and able to bind TNF with such an affinity that the interaction with the membrane-bound TNF receptor is inhibited or blocked, in a preferred embodiment, human soluble TNF-RI is the TNF antagonist to be administered to patients. The natural and recombinant soluble TNF receptor molecules and methods of their production have been described in the European Patent Applications EP 308,378; EP 398,327 and EP 433,900 and U.S. Pat. Nos. 5,359,037; 5,512,544; 5,695,953; 5,811,261; 5,981,701; 6,232,446; 6,262,239; and US20010019833A1.

TNF receptor multimeric molecules and TNF immunoreceptor fusion molecules, and derivatives or portions thereof, are additional examples of receptor molecules useful in the methods of the present invention. TNF receptor multimeric molecules useful in the present invention comprise all or a functional portion of the extracellular domain of two or more TNF receptors linked via one or more polypeptide linkers. The multimeric: molecules can further comprise a signal peptide of a secreted protein to direct expression of the multimeric molecule.

These multimeric molecules and methods of their production have been described in the European Patent Application EP 526,905.

TNF immunoreceptor fusion molecules useful in the methods of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. TNF immunoreceptor fusion molecules and methods for their production have been described in the European Patent Application EP 620,739, corresponding to PCT Patent Application WO 94/06476.

Another class of sequestering antagonists useful in the method of the present invention is represented by the anti-TNF antibodies, including monoclonal, chimeric humanized, and recombinant antibodies and fragment thereof which are characterized by high affinity binding to TNF in vivo and low toxicity. The antibodies which can be used in the invention are characterized by their ability to treat patients for a hormone dependent breast cancer and low toxicity. Neutralizing antibodies are readily raised in animals such as rabbits or mice by immunization with TNF.

Immunized mice are particularly useful for providing sources of B cells for the manufacture of hybridomas, which in turn are cultured to produce large quantities of anti-TNF monoclonal antibodies. Chimeric antibodies are immunoglobulin molecules characterized by two or more segments or portions derived from different animal species. Generally, the variable region of the chimeric antibody is derived from a non-human mammalian antibody, such as murine monoclonal antibody, and the immunoglobulin constant region is derived from a human immunoglobulin molecule. Preferably, both regions and the combination have low immunogenicity as routinely determined (Elliott et al., “Randomised Double-blind Comparison of Chimeric Monoclonal Antibody to Tumour Necrosis Factor alpha (cA2) versus Placebo in Rheumatoid Arthritis”, Lancet, 344:1105-1110 (1994). Humanized antibodies are immunoglobulin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans (Knight et al., Construction and initial characterization of a mouse-human chimeric anti-TNF antibody. Mol Immunol. 1993 November;30(16):1443-53). Preferred examples of high affinity monoclonal antibodies and chimeric derivatives thereof, useful in the methods of the present invention, are described in the European Patent Application EP186,833; PCT Patent Application WO 92/16553; and U.S. Pat. No. 6,090,923.

TNF antagonist can be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the TNF antagonist is administered to the patient (e.g. via a vector) which causes the TNF antagonist to be expressed and secreted in vivo. In addition, the TNF antagonist can be administered together with other components of biologically active agents such as pharmaceutically acceptable surfactants, excipients, diluents or any other carrier.

Techniques for obtaining, manipulating and expressing cloned genes can be used to make variants and analogs of TNF antagonists such as soluble TNF receptors and other proteins useful in the invention. Recombinant DNA methods, chemical synthetic methods, enzymatic methods and mixed methods for making, altering and utilizing muteins and peptide analogs are well known. For instance, such methods are set forth in Sambrook and Russel, MOLECULAR CLONING: A LABORATORY MANUAL, 3rd Ed., Cold Spring Harbor Laboratory Press, Cold Spring Harbor, N.Y. (2001), the entirety of which is herein incorporated by reference.

It will be appreciated by those of skill that cloned genes readily can be manipulated to alter the amino acid sequence of a protein. The cloned gene for human TNF receptor can be manipulated by a variety of well known techniques for in vitro mutagenesis, among others, to produce variants of the naturally occurring human protein, herein referred to as muteins, that may be used in accordance with the invention.

Classes of IL-1 antagonists include interleukin-1 receptor antagonists (any compound capable of specifically preventing activation of cellular receptors to IL-1) such as IL-1ra, as described below; anti-IL-1 receptor monoclonal antibodies (e.g., EP 623674), the disclosure of which is hereby incorporated by, reference; IL-1 binding proteins such as soluble IL-1 receptors (e.g., U.S. Pat. Nos. 5,492,888, 5,488,032, and U.S. Pat. NoS. 5,464,937, 5,319,071, and U.S. Pat. No. 5,180,812); anti-IL-1 monoclonal antibodies (e.g., WO 9501997, WO 9402627, WO 9006371, U.S. Pat. No. 4,935,343, EP 364778, EP 267611 and EP 220063); IL-1 receptor accessory proteins (e.g., WO 96/23067), and other compounds and proteins which block in vivo synthesis or extracellular release of IL-1.

IL-1 receptor antagonist (IL-1ra) is a human protein that acts as a natural inhibitor of IL-1. Preferred receptor antagonists, as well as methods of making and methods of using thereof, are described in U.S. Pat. No. 5,075,222; WO 91/08285; WO 91/17184; AU 9173636; WO 92/16221; WO 93/21946; WO 94/06457; WO 94/21275; FR 2706772; WO 94/21235; DE 4219626; WO 94/20517; WO 96/22793 and WO 97/28828. The proteins include glycosylated as well as non-glycosylated IL-1 receptor antagonists.

Specifically, three preferred forms of IL-1ra (IL-1ra.alpha., IL-1ra.beta. and IL-1rax), each being derived from the same DNA coding sequence, are disclosed and described in U.S. Pat. No. 5,075,222. Methods for producing IL-1 antagonists, particularly IL-1ras, are also disclosed in the U.S. Pat. No. 5,075,222 patent. In a specific embodiment, an IL-1ra contains an N-terminal methionyl group as a consequence of expression in E. coli. The present invention also includes modified IL-1ras. The modified IL-1ras include, for example, muteins of such inhibitors in which a cysteine residue is substituted for an amino acid at one or more sites in the amino acid sequence of a naturally-occurring inhibitor. Such muteins may then be site-selectively reacted with fictionalized polyethylene glycol (PEG) units or other sulfhydryl-containing polyethers to create IL-1ra PEG species. WO 92/16221 discloses a number of modified IL-1ra species and methods of making such PEG modified inhibitors.

An additional class of IL-1 antagonists includes compounds capable of specifically preventing activation of cellular receptors to IL-1. Such compounds include IL-1 binding proteins, such as soluble receptors and monoclonal antibodies. Such compounds also include monoclonal antibodies to the receptors.

A further class of interleukin-1 antagonists includes compounds and proteins which block in vivo synthesis and/or extracellular release of IL-1. Such compounds include agents which affect transcription of IL-1 genes or processing of IL-1 preproteins.

The variation in primary structure of muteins of TNF and IL-1 receptors useful in the invention, for instance, may include deletions, additions and substitutions. The substitutions may be conservative or non-conservative. The differences between the natural protein and the mutein generally conserve desired properties, mitigate or eliminate undesired properties and add desired or new properties. In the present invention the muteins generally are those that maintain or increase anti-TNF or anti-IL-1 activity. Particularly, TNF and IL-1 receptor muteins are amino acid sequence variants of the TNF and IL-1 receptors that maintain or increase the inhibition of estradiol induction.

Similarly, techniques for making small oligopeptides and polypeptides that exhibit activity of larger proteins from which they are derived (in primary sequence) are well known and have become routine in the art. Thus, peptide analogs of proteins of the invention, such as peptide analogs of soluble TNF or IL-1 receptors that exhibit TNF or IL-1 antagonist activity, respectively, also are useful in the invention.

Mimetics also can be used in accordance with the present invention to prevent or treat breast cancer. The design of mimetics is known to those skilled in the art, and is generally understood to be peptides or other relatively small molecules that have an activity the same or similar to that of a larger molecule, often a protein, on which they are modeled.

Compounds other than the proteins, muteins, protein-derived mimetics and the like discussed above that inhibit the TNF induced estradiol production in adipocytes also may be useful in the present invention. Among such compounds are certain small organic molecules, which may be mimetics. Examples of such small molecules include, but are not limited to, PCM (Omega Pharmaceuticals Inc.), SH-636 (Shering AG, Germany), NPI-1302 (Hensler et al. abstract in Proceedings: American Association for Cancer Research, Cytokines and Cancer: Regulation, Angiogenesis, and Clinical Applications; Vail, Colorado, Sep. 20-24, 2000); and 1515-104838 (Isis Pharmaceuticals, Inc., Carlsbad, Calif.).

All forms of human IFN-γ that are shown to be biologically active can be used according to the present invention. These forms include mature, pro, met and/or des(1-3) (also referred to as des-Cys-Tyr-Cys IFN-γ) form, whether obtained from natural source, chemically synthesized or produced by techniques of recombinant DNA technology. A complete description of the preparation of recombinant human IFN-γ (rhu IFN-γ) including its cDNA and amino acid sequences are disclosed, for example, in U.S. Pat. Nos. 4,727,138; 4,762,791; 4,925,793; 4,929,554; 5,582,824; 5,096,705; 4,855,238; 5,574,137; and U.S. Pat. No. 5,595,888. CysTyrCys-lacking recombinant human IFN-γ, including variously truncated derivatives are, for example, disclosed in U.S. Pat. No. 5,582,824. IFN-γ usefull in the present invention includes variously glycosylated forms and other variants (e.g. amino acid sequence variants) and derivatives of such native (wild-type) IFN-γ, whether known in the art or becoming available in the future. Examples of such variants are alleles, and the products of site-directed mutagenesis in which residues are deleted, inserted and/or substituted (see, e.g. U.S. Pat. No. 5,582,824 referred to above). IFN-γ useful in the present invention is available from a wide variety of commercial sources and it is approved for the treatment of numerous indications.

The IFN-γ used according to the present invention may be from natural sources, but is preferably a recombinant product. IFN-γ useful according to the present invention also includes polypeptides or fragments thereof which have IFN-γ activity, and chimeric or mutant forms of IFN-γ in which sequence modifications have been introduced, for example to enhance stability, without affecting the nature of their biological activity, such as disclosed in U.S. Patent Nos. U.S. Pat. No. 5,593,667, and U.S. Pat. No. 5,594,107 among others. For example, the IFN-γ useful in the present invention can be a recombinant human IFN-γ species (recombinant human interferon gamma-1b, rh IFN-γ-1b, containing 140 amino acids), which is the active ingredient of the commercial formulation, Actimmune®D (Genentech, Inc., South San Francisco, Calif.).

The definition of “pharmaceutically acceptable” is meant to encompass any carrier, which does not interfere with effectiveness of the biological activity of the active ingredient and that is not toxic to the host to which it is administered. For example, for parenteral administration, INF antagonist, an IL-1 antagonist and/or IFN-γ may be formulated in a unit dosage form for injection in vehicles such as saline, dextrose solution, serum albumin and Ringer's solution.

For parenteral (e.g. intravenous, subcutaneous, intramuscular) administration, TNF antagonists, IL-1 antagonists and/or IFN-γ can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective estradiol inhibiting amount of a TNF antagonist, an IL-1 antagonist and/or IFN-γ will be a function of many variables. The variables include the type of TNF or IL-1 antagonist, the affinity of the antagonist for TNF or IL-1, any residual cytotoxic activity exhibited by the antagonists, the route of administration, the clinical condition of the patient (including the desirability of maintaining a non-toxic level of endogenous TNF activity, IL-1 activity or IFN-γ activity), and the presence of multiple INF or IL-γ combining sites in sequestering agents, e.g. antibodies.

A “therapeutically effective estradiol inhibiting amount” is such that when administered, the TNF antagonist, the IL-1 antagonist or IFN-γ results in a decrease in estradiol production from adipocytes. A 50% decrease is preferred. More preferably the decrease in estradiol production is 75%, most preferably 95%. The dosage administered, as single or multiple doses, to an individual will vary depending upon a variety of factor, including compounds pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled, as well as in vitro and in vivo methods of determining the inhibition of estradiol.

In patients having a risk factor for breast cancer, for example, family history of the disease or genetic factors such as mutations within the BRCA1 and BRACA2 genes, the TNF antagonist, IL-1 antagonist and/or IFN-γ can be administered prophylactically either separately, simultaneously, or sequentially with other anti-estrogenic therapies for the prevention of breast cancer. If IFN-γ, TNF antagonists or IL-1 antagonists are administered simultaneously with other therapeutic agents, they can be administered in the same or different composition.

Examples of the anti-estrogenic agents include, but are not limited to 2-phenyl-3-benzothiophenes and 1-alkylaminoethoxy phenyl)-1-phenyl-2-phenylbut-1-enes represented by raloxifene and tamoxifen; 4-hydroxytamoxifen; clomiphene; nafoxidine (Upjohn & Co., 700 Portage Road, Kalamazoo, Mich.); non-steroidal sulfatase inhibitor compounds as described in U.S. Pat. No. 5,567,831; derivatives of estra 1,3,5 (10)triene-17-one, 3-amino compounds as in U.S. Pat. No. 5,571,933; anti-estrogenic steroid sulfatase inhibitors as described in U.S. Pat. No. 6,288,050. Other anti-estrogenic agents that are contemplated in the present invention are toremifene, droloxifene, TAT-59, idoxifene, EM 139, clomiphene, MER-25, DES, nafoxidene, CP-336,156, GW5638, LY139481, LY353581, zuclorniphene, enclomiphene, ethamoxytriphetol, delmadinone acetate, bisphosphonate, and the like.

For example, treatment of breast cancer using raloxifene has been described in the U.S. Pat. No. 4,418,068. Droloxifene (3-hydroxytamoxifen) has been reported to be effective in the treatment of breast cancer U.S. Pat. No. 5,047,431.

Several steroidal anti-estrogens have been synthesized which lack estrogenic activity. Included among these are ICI 164,384, ICI 182,780 and RU 58668. See, e.g.: Wakeling et al. J Steroid Biochem. 31:645-653 (1988), which pertains to ICI 164,384; Wakeling et al., Cancer Res. 51:3867-3873 (1991), and Wakeling et al., J. Steroid Biochem. Molec. Biol. 37:771-774 (1990), which pertain to ICI 182,780; and Van de Velde et al., Ann. N.Y. Acad. Sci. 761:164-175 (1995), Van de Velde et al., Pathol. Biol 42:30 (1994), and Nique et al., Drugs Future 20:362-366 (1995), which relate to RU 58668.

Anti-estrogenic agents as provided in the U.S. Pat. No. 6,281,205 are also contemplated in the present invention. Anti-estrogenic agents useful in the present invention also include estrogen receptor modulators as described, for example in U.S. Pat. No. 6,300,367 including, but not limited to raloxifene, droloxifene, toremifene, 4′-iodotamoxifen, and idoxifene is co-administered with at least one isoflavone selected from genistein, daidzein, biochanin A, formononetin, and their naturally occuring glucosides and glucoside conjugates. In addition, known aromatase inhibitors are contemplated as anti-estrogenic agents. Examples of such aromatase inhibitors include formula (1) in International patent application publication No. WO 94/13645 such as 1-[1-(4-cyanophenyl)-3-(4-fluorophenyl)-2-hydroxypropyl]-1,2,4-triazole, diasteroisomer a+d, which also is known under the code MPV-2213ad. Other suitable aromatase inhibitors include, but are not limited to aminoglutethimide, anastrozole, CGS 16949A, 4-hydroxyandrostenedione (4-OHA), fadrozole, letrozole, vorozole, roglethimide, atamestane, exemestane, formestane, YM-511 (4-[N-(4-bromobenzyl)-N-(4-cyanophenyl)amino]-4H- 1,2,4-triazole), ZD-1033 (arimedex) and NKS-01 (14-alpha-hydroxyandrost-4-ene-3,6,17-trione) and their stereoisomers.

The invention also features an article of manufacture including packaging material and a pharmaceutical agent contained therein that is therapeutically effective for treating or preventing estrogen-dependent breast cancer in an individual. The packaging material may include a label that indicates that the pharmaceutical agent can be used for treating and/or preventing estrogen-dependent breast cancer. The pharmaceutical agent includes an effective amount of IFN-α or an effective amount of a TNF antagonist or an effective amount of IL-1 antagonist. In one embodiment, the pharmaceutical agent includes a combination of an effective amount of IFN-γ and a TNF antagonist. In yet another embodiment, the pharmaceutical agent includes a combination of an effective amount of IFN-γ and a TNF antagonist and an IL-1 antagonist.

In an alternate embodiment, the invention relates to compositions and kits comprising a first estrogen-dependent cancer treating or preventing agent including IFN-γ and/or a TNF antagonist and/or an IL-1 antagonists and a second therapeutic agent. The second therapeutic agent is not IFN-γ or a TNF antagonist or an IL-1 antagonist. These compositions are effective to treat or prevent estrogen-dependent breast cancer in an individual. Various classes of therapeutic agents, including anti-estrogenic agents such as tamoxifen, may be used in the composition. In one embodiment, the first estrogen-dependent cancer treating or preventing pharmaceutical agent includes a combination of an effective amount of IFN-γ and a TNF antagonist. In another embodiment, the first estrogen-dependent cancer treating or preventing pharmaceutical agent includes a combination of an effective amount of IFN-γ and a TNF antagonist and an IL-1 antagonist.

The present invention will now be illustrated by the example, which is not intended to be

limiting in anyway, and makes reference to the following figures.

### EXAMPLE 1

Human subcutaneous cultured adipocytes and preadipocytes, catalog nos. SP-1012, SP-1024, SP-1096, SP-75, or SP-25 were purchased from Zen-Bio, Inc. (Research Triangle Park, N.C.) and cultured according to the manufacturer's instructions.

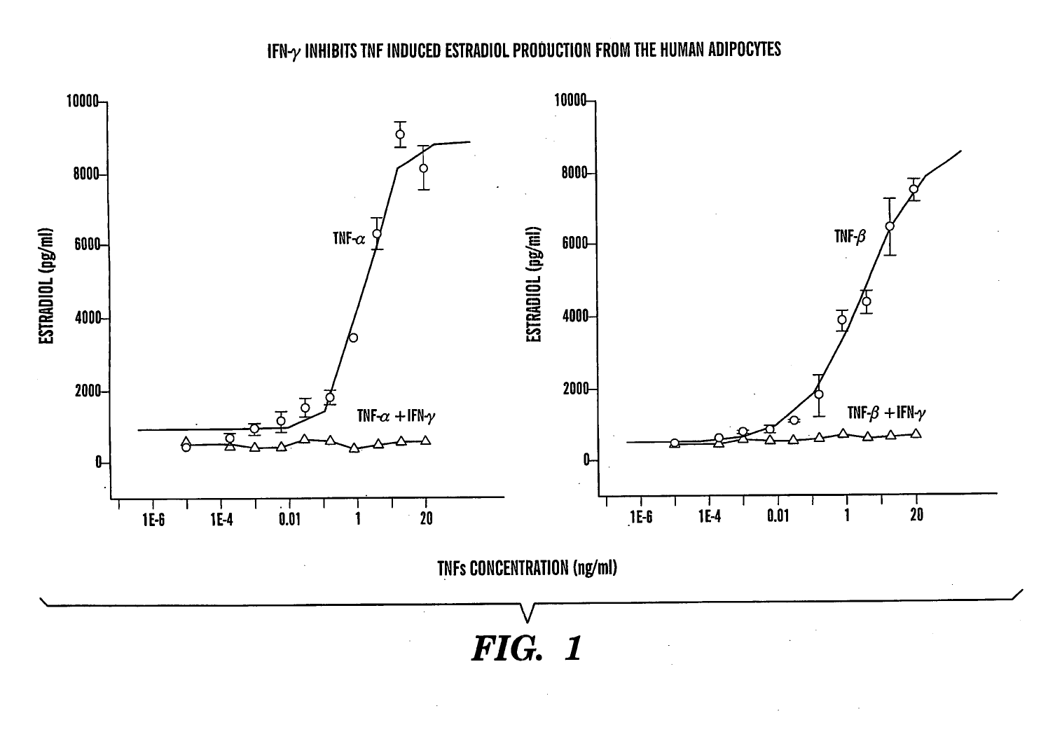

The culture medium of the preadipocytes was supplemented with 0.1 μg/ml IFN-γ and increasing concentrations of either TNF-α or TNF-β as shown in FIG. 1. Estradiol concentrations were measured using the Active™ Estradiol EIA kit according to the manufacturer's instructions (catalog no. DSL-10-4300, Diagnostic Systems Laboratories, Inc., Webster, Tex.). As indicated by filled ovals in FIG. 1, both TNF-α and INF-β, significantly increased estradiol amount. However, when IFN-γ was given to the cells, the filled triangles show that interferon gamma (IFN-γ) inhibits tumor necrosis factor alfa (TNF-α) and tumor necrosis factor beta (TNF-β) induced estradiol production in human adipocytes.

### EXAMPLE 2

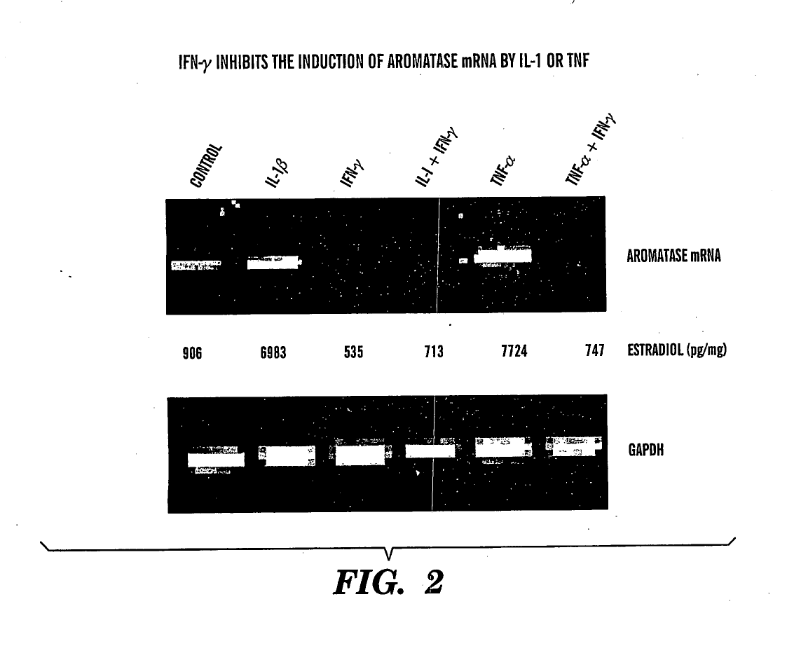

Adipocyte cultures were treated with IL-1-β, IFN-γ, TNF-α, and combinations of IL-1-β and IFN-γ and TNF-α and IFN-γ. Consequently, total RNA from cultured adipocytes was isolated using conventional methods. The reverse transcriptase polymerase chain reaction (RT-PCR) was performed using aromatase specific primers. The amplified aromatase cDNA fragments produced by RT-PCR were separated on an agarose gel. As shown in the FIG. 2, IL-1β and TNF-α alone induced transcription of aromatase mRNA compared to untreated control. However, the administration of IFN-γ inhibits the transcription of aromatase mRNA when given together with IL-1β or TNFα. The lower panel shows transcription of a constitutively expressed household gene, glyceraldehyde-phosphate dehydrogenase (GAPDH). Control lane shows the base level of aromatase mRNA expression in adipocytes. The estradiol production by the cells is shown as pikograms/ml (pg/ml) below the photograph of the gel showing the aromatase mRNA levels.

### EXAMPLE 3

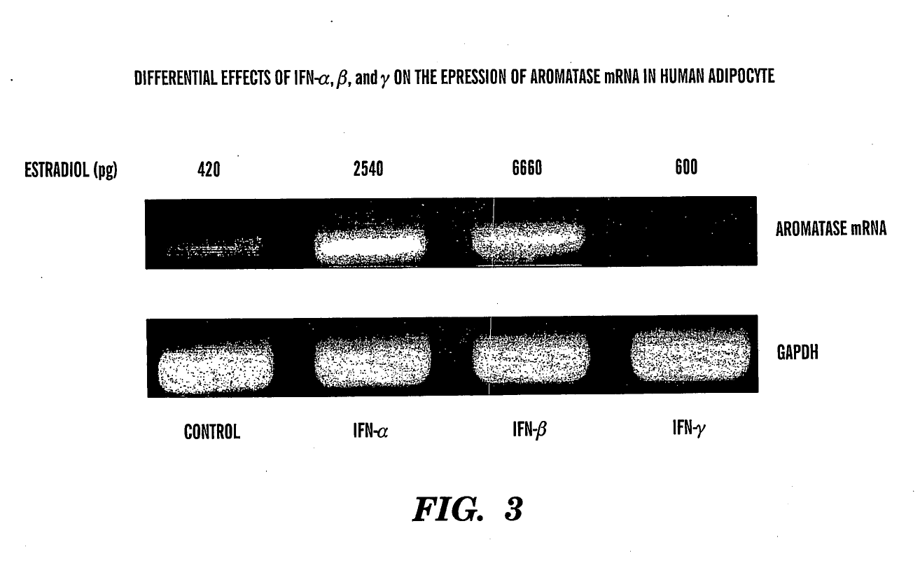

The isolated human adipocytes were also treated with different interferon family members: IFN-α, -β, and -γ. As shown in FIG. 3, the expression of aromatase MRNA in human adipocytes was significantly increased by addition of IFN-α and -β whereas IFN-γ inhibits the endogenous aromatase MRNA expression. The estradiol production increased by about 6 fold upon IFN-A induction and by about 16 fold upon IFN-β induction (shown in pg estradiol/ml of culture medium above the gel photograph showing the aromatase mRNA levels).

### EXAMPLE 4

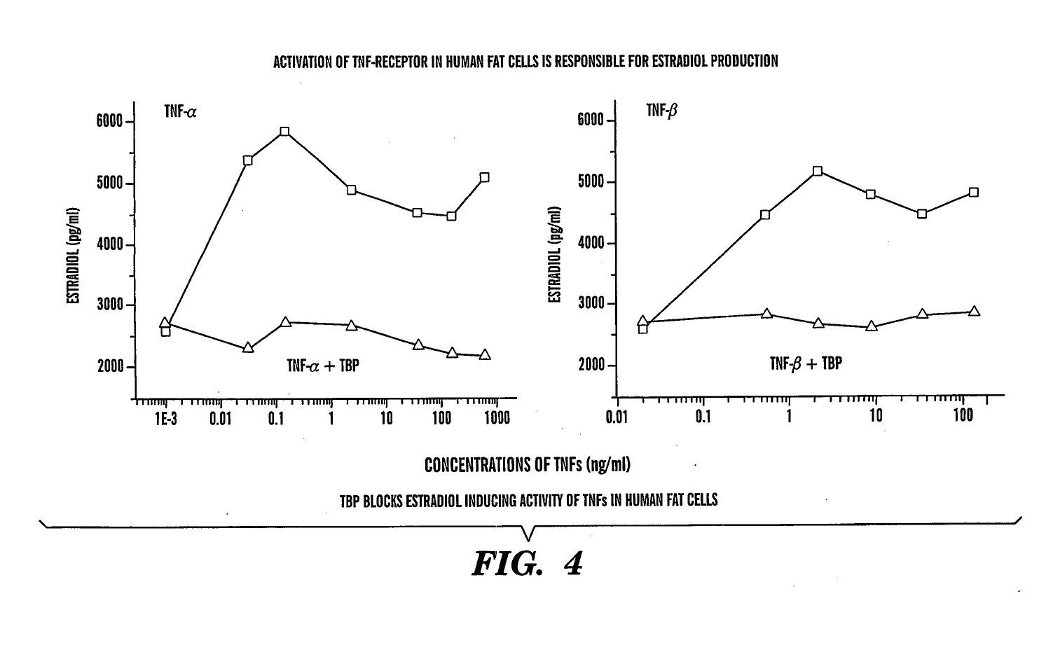

The isolated human adipocytes were grown in the presence of TNF-α and TNF-β at varying concentrations as indicated in the FIG. 4 with filled squares. When the adipocyte cultures were grown in the presence of both TNF-α and soluble TNF receptor, TNF-R1 as described in U.S. Pat. No. 6,225,300 (TBP) or, in a parallel wells, in the presence of TNF-β and TBP, the production of estradiol was completely blocked as shown with filled triangles in the FIG. 4.

The preceding examples are to be evaluated as illustrative and are not intended to limit the scope of this invention.

All publications and patent applications cited in this specification are herein incorporated by reference as if each individual publication or patent application were specifically and individually indicated to be incorporated by reference. Although the foregoing invention has been described in some detail by way of illustration and an example for purposes of clarity of understanding, it will be readily apparent to those of ordinary skill in the art in light of the teachings of this invention that certain changes and modifications may be made thereto without departing from the spirit or scope of the appended claims.

1. A method of treating estrogen responsive breast cancer in an individual comprising administering a therapeutically effective estradiol inhibiting amount of IFN-γ.

2. A method of preventing estrogen responsive breast cancer in an individual in a risk of the cancer comprising administering a therapeutically effective estradiol inhibiting amount of IFN-γ.

3. A method of treating estrogen responsive breast cancer in an individual comprising administering a therapeutically effective estradiol inhibiting amount of a TNF antagonist.

4. A method of preventing estrogen responsive breast cancer in an individual in a risk of the cancer comprising administering a therapeutically effective estradiol inhibiting amount of a TNF antagonist.

5. A method of treating estrogen responsive breast cancer in an individual comprising administering a therapeutically effective estradiol inhibiting amount of a combination of IFN-γ and a TNF antagonist.

6. A method of preventing estrogen responsive breast cancer in an individual in a risk of the cancer comprising administering a therapeutically effective estradiol inhibiting amount of a combination of IFN-γ and a TNF antagonist.

7. A method of treating estrogen responsive breast cancer in an individual comprising administering a therapeutically effective estradiol inhibiting amount of an IL-1 antagonist.

8. A method of preventing estrogen responsive breast cancer in an individual in a risk of the cancer comprising administering a therapeutically effective estradiol inhibiting amount of an IL-1 antagonist.

9. A method of treating estrogen responsive breast cancer in an individual comprising administering a therapeutically effective estradiol inhibiting amount of IFN-γ in combination with an anti-estrogenic agent.

10. A method of preventing estrogen responsive breast cancer in an individual in a risk of the cancer comprising administering a therapeutically effective estradiol inhibiting amount of IFN-γ in combination with an anti-estrogenic agent.

11. A method of treating estrogen responsive breast cancer in an individual comprising administering a therapeutically effective estradiol inhibiting amount of a TNF antagonist in combination with a simultaneous, sequential, or separate administration of an anti-estrogenic agent.

12. A method of preventing estrogen responsive breast cancer in an individual in a risk of the cancer comprising administering a therapeutically effective estradiol inhibiting amount of a TNF antagonist in combination with a simultaneous, sequential, or separate administration of an anti-estrogenic agent.

13. A method of treating estrogen responsive breast cancer in an individual comprising administering a therapeutically effective estradiol inhibiting amount of IFN- and a TNF antagonist in combination with a simultaneous, sequential, or separate administration of an anti-estrogenic agent.

14. A method of preventing estrogen responsive breast cancer in an individual in a risk of the cancer comprising administering a therapeutically effective estradiol inhibiting amount of IFN-γ and a TNF antagonist in combination with a simultaneous, sequential, or separate administration of an anti-estrogenic agent.

15. The method of claim 9, 10, 11, 12, 13, or 14, wherein the anti-estrogenic agent is tamoxifen.

16. Cancelled

17. Cancelled

18. Cancelled

19. Cancelled

20. Cancelled

20. Cancelled

21. A kit comprising:
i) a packaging material comprising a label which indicates said pharmaceutical may be administered, for a sufficient term at an effective dose, for treating or preventing estrogen responsive breast cancer in an individual; and ii) a pharmaceutical agent contained within said packaging material, wherein said pharmaceutical agent comprisesIFN-γ together with a pharmaceutical acceptable carrier

22. A kit comprising:
i) a packaging material comprising a label which indicates said pharmaceutical may be administered, for a sufficient term at an effective dose, for treating or preventing estrogen responsive breast cancer in an individual; and
ii) a pharmaceutical agent contained within said packaging material, wherein said pharmaceutical agent comprises a TNF antagonist together with a pharmaceutical acceptable carrier

23. A kit comprising:
ii) a packaging material comprising a label which indicates said pharmaceutical may be administered, for a sufficient term at an effective dose, for treating or preventing estrogen responsive breast cancer in an individual; and
ii) a pharmaceutical agent contained within said packaging material, wherein said pharmaceutical agent comprises a combination of IFN-γ and a TNF antagonist together with a pharmaceutical acceptable carrier

24. The method of claims 3, 4, 5, 6, 9, 10, 11 or 12, wherein the TNF antagonist is selected from the group consisting of a tumor necrosis binding protein, a soluble TNF receptor molecule, or an anti-TNF antibody.

25. The method of claim 1, 2, 3,4, 5, 6, 7, 9, 10, 11, 12, 13, or 14 further comprising administering of therapeutically effective amount of an IL-1 antagonist.

26. The method of claim 7 or 8 further comprising administering a therapeutically effective amount of IFN-γ and/or a TNF antagonist.

27. A kit comprising:
i) a packaging material comprising a label which indicates said pharmaceutical may be administered, for a sufficient term at an effective dose, for treating or preventing estrogen responsive breast cancer in an individual; and
ii) a pharmaceutical agent contained within said packaging material, wherein said pharmaceutical agent comprises an IL-1 antagonist together with a pharmaceutical acceptable carrier

28. The kit of claim 21, 22, or 23 wherein said pharmaceutical agent further comprises an IL-1 antagonist.
